(19) United States
(12) Patent Application Publication
(10) Pub. No.: US 2017/0151104 A1
(43) Pub. Date: Jun. 1, 2017

(54) ABSORBENT ARTICLE WITH FLUID ACQUISITION LAYER HAVING CHANNELS

(71) Applicant: Kimberly-Clark Worldwide, Inc., Neenah, WI (US)

(72) Inventors: Lisha Yu, Appleton, WI (US); Yung Huang, Appleton, WI (US); Andrew T. Hammond, Grand Chute, WI (US); Russell J. Brumm, Menasha, WI (US)

(21) Appl. No.: 15/309,658

(22) PCT Filed: May 29, 2014
(86) PCT No.: PCT/US14/39997

§ 371 Date: Nov. 8, 2016

(51) Int. Cl. A61F 13/537

(52) U.S. Cl. CPC A61F 13/53747; A61F 13/53717; A61F 2013/53782

(57) ABSTRACT

An absorbent article can include a body facing liner, a back-sheet coupled to the body facing liner, and an absorbent body. The absorbent article can also include a fluid acquisition layer. The fluid acquisition layer can include a plurality of apertures and at least one channel. The channel can include a length and a depth. The length can be defined in a horizontal plane by a distance between a proximal end and a distal end. The proximal end can be connected to a first aperture and the depth can extend from a body facing surface of the fluid acquisition layer to a garment facing surface of the fluid acquisition layer.

### TECHNICAL FIELD

The present invention relates to absorbent articles.

### BACKGROUND OF THE DISCLOSURE

One of the primary functions of personal care absorbent articles is to retain and absorb body exudates such as urine, fecal material, blood, and menses. Along these lines, a desired attribute of personal care absorbent articles is to minimize the leakage of such exudates from the absorbent article. It is also desired, however, that personal care absorbent articles retain and absorb the body exudates in such a fashion so as to provide a dry feel to the wearer, removing exudates from against the skin at the time of the initial insult of the exudate as well as retaining them away from the skin after such insult.

Absorbent articles, however, traditionally fail to possess the combination of the desired attributes. Absorbent articles commonly fail before the total absorbent capacity of the absorbent article is utilized. Problems which can typically exist can relate to the ability of the body facing liner and other components of the absorbent article to allow quick intake in one direction towards an absorbent body while preventing return of fluid in the opposite direction. Additionally, the rate at which intake occurs sometimes determines whether leakage is reduced or whether body fluids are appropriately contained.

Especially troublesome can be semi-solid fecal material, such as low viscosity fecal material which can be prevalent with younger children, and menses. Such body exudates have difficulty penetrating the body facing material of the absorbent article as easily as low viscosity exudates, such as urine, and tend to spread across the surface of the body facing material. And once these exudates do penetrate the body facing material, the exudates may have trouble penetrating other components of the absorbent article, such as fluid acquisition layers or fluid transfer layers, to be absorbed by the absorbent body. As a result of the inefficient penetration of the exudates to the absorbent body, these exudates may move back to the surface of the body facing material and against the wearer's skin, which can increase the potential for skin irritation and can lead to smears against the skin of the wearer making clean-up of the skin difficult.

Thus, there remains a need for an absorbent article that can provide increased efficiency of intake of body exudates into the absorbent body and improved handling of body exudates to minimize the amount of body exudates in contact with the wearer's skin.

### SUMMARY OF THE DISCLOSURE

In one embodiment, the invention provides for an absorbent article including a longitudinal axis and a lateral axis. The longitudinal axis and the lateral axis are defined in a horizontal plane parallel to the absorbent article in a laid flat configuration. The absorbent article can also include a vertical axis perpendicular to the horizontal plane. The absorbent article can include a body facing liner; a backsheet coupled to the body facing liner, and an absorbent body positioned between the body facing liner and the backsheet. The absorbent article can also include a fluid acquisition layer that can include a body facing surface and a garment facing surface. The fluid acquisition layer can be positioned between the body facing liner and the backsheet. The fluid acquisition layer can further include a plurality of apertures and at least one channel including a length and a depth. The length can be defined in the horizontal plane by a distance between a proximal end and a distal end of the channel and the depth can be defined in a vertical direction parallel to the vertical axis. The proximal end can be connected to a first aperture of the plurality of apertures. The at least one channel can be configured such that the depth extends from the body facing surface to the garment facing surface.

In another embodiment, the invention provides for an absorbent article including a longitudinal axis and a lateral axis. The longitudinal axis and the lateral axis are defined in a horizontal plane parallel to the absorbent article in a laid flat configuration. The absorbent article can also include a vertical axis perpendicular to the horizontal plane. The absorbent article can include a body facing liner; a backsheet coupled to the body facing liner, and an absorbent body positioned between the body facing liner and the backsheet. The absorbent article can also include a fluid acquisition layer. The fluid acquisition layer can be positioned between the body facing liner and the absorbent body. The fluid acquisition layer can include a first layer and a second layer. The first layer can include a top surface providing a body facing surface of the fluid acquisition layer and an opposing bottom surface. The second layer can include a bottom surface providing a garment facing surface of the fluid acquisition layer and an opposing top surface. The fluid acquisition layer can further comprise a plurality of apertures and at least one channel including a length and a depth. The length can be defined in the horizontal plane by a distance between a proximal end and a distal end of the channel and the depth can be defined in a vertical direction parallel to the vertical axis. The proximal end can be connected to a first aperture of the plurality of apertures. The at least one channel can be configured such that the depth extends through at least one of the first layer and the second layer.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a side perspective view of an exemplary embodiment of an absorbent article, such as a diaper, in a fastened condition.

FIG. 2 is a top plan view of the absorbent article of FIG. 1 in an unfastened, stretched, and laid flat condition with the body facing surface of the absorbent article which contact the wearer facing the viewer, portions of the absorbent article being cut away for clarity of illustration.

FIG. 3 is a top plan view of an acquisition layer from the absorbent article of FIG. 1, the acquisition layer having a plurality of apertures and a plurality of channels.

FIG. 4A is a cross-sectional view of the acquisition layer of FIG. 3 taken along line 4-4.

FIG. 4B is a cross-sectional view of an alternative embodiment of an acquisition layer having channels with varying depth along the length of the channels.

FIG. 5A is a cross-sectional view of an alternative embodiment of an acquisition layer including two layers, with a plurality of apertures and channels each being provided in the first layer.

FIG. 5B is a cross-sectional view of an alternative embodiment of an acquisition layer including two layers, with a plurality of apertures and channels each being provided in the second layer.

FIG. 5C is a cross-sectional view of an alternative embodiment of an acquisition layer including two layers, with a plurality of apertures extending through both the first and second layer and channels in both the first and second layer that only extend through the layer in which they are provided.

FIG. 5D is a cross-sectional view of an alternative embodiment of an acquisition layer including two layers, with a plurality of apertures and channels extending through both the first and second layers.

FIG. 6 is a top plan view of an alternative embodiment of an acquisition layer, the acquisition layer having a plurality of apertures and a plurality of channels.

FIG. 7 is a top plan view illustrating an exemplary alignment of a body facing liner having a plurality of intersecting slit formations and an acquisition layer having a plurality of apertures and a plurality of channels, the body facing liner overlaying the acquisition layer facing the viewer.

FIG. 8 is a detailed view taken along line 8-8 in FIG. 7.

FIG. 9 is a top plan view of an exemplary embodiment of an absorbent article, such as a feminine hygiene product.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards an absorbent article that can have an acquisition layer with at least one channel. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

Definitions:

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products, including, but not limited to, menstrual pads, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. The bonding of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” refers herein to one who fits an absorbent article, such as, but not limited to, a diaper, training pant, youth pant, incontinent product, or other absorbent article about the wearer of one of these absorbent articles. A user and a wearer can be one and the same person.

Absorbent Article:

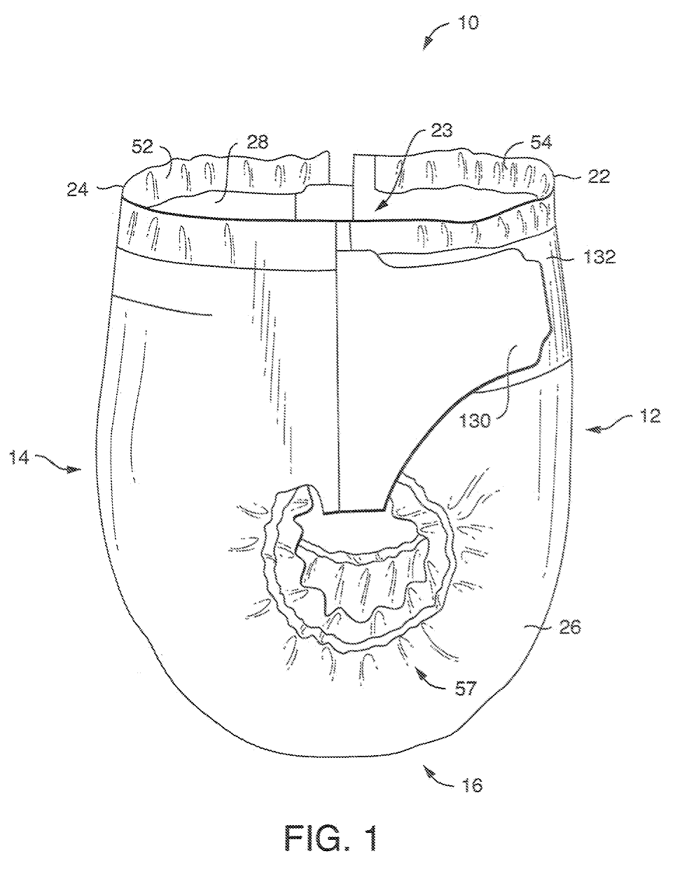

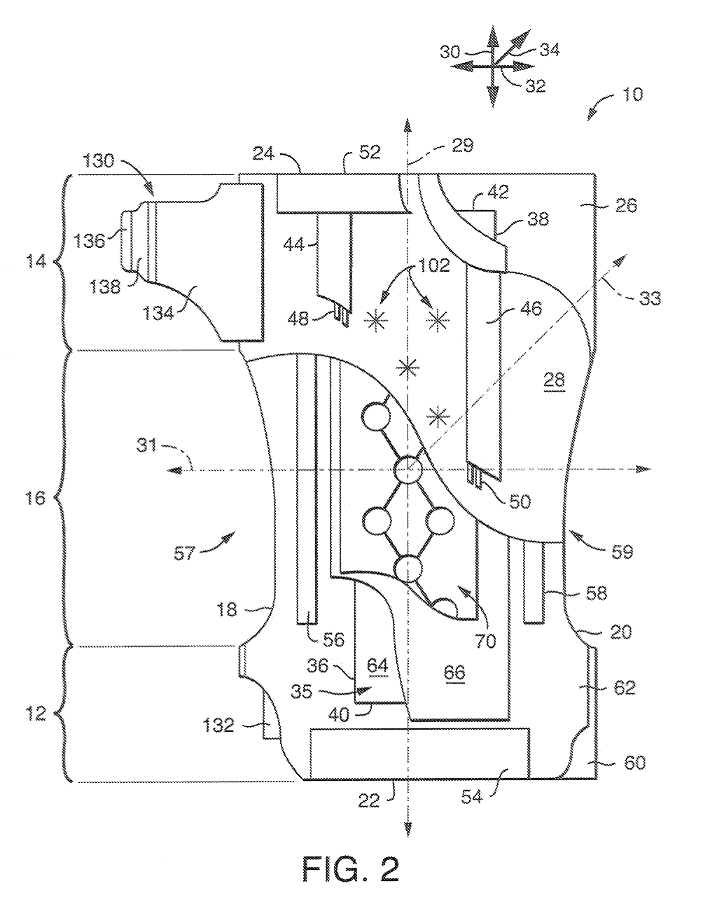

Referring to FIGS. 1 and 2, a non-limiting illustration of an absorbent article 10, for example, a diaper, is illustrated. Other embodiments of the absorbent article could include training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure. The absorbent article 10 illustrated in FIGS. 1 and 2 includes a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front end region, the rear waist region 14 can be referred to as the rear end region, and the crotch region 16 can be referred to as the intermediate region. The absorbent article 10 has a pair of longitudinal side edges, 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24.

The front waist region 12 can include the portion of the absorbent article 10 that, when worn, is positioned at least in part on the front of the wearer while the rear waist region 14 can include the portion of the absorbent article 10 that, when worn, is positioned at least in part on the back of the wearer. The crotch region 16 of the absorbent article 10 can include the portion of the absorbent article 10, that, when worn, is positioned between the legs of the wearer and can partially cover the lower torso of the wearer. The waist edges, 22 and 24, of the absorbent article 10 are configured to encircle the waist of the wearer and together define the central waist opening 23. Portions of the longitudinal side edges, 18 and 20, in the crotch region 16 can generally define leg openings when the absorbent article 10 is worn.

The absorbent article 10 can include a backsheet 26 and a body facing liner 28. In an embodiment, the body facing liner 28 can be bonded to the backsheet 26 in a superposed relation by any suitable means such as, but not limited to, adhesives, ultrasonic bonds, thermal bonds, pressure bonds, or other conventional techniques. The backsheet 26 can define a length in a longitudinal direction 30, and a width in the lateral direction 32, which, in the illustrated embodiment, can coincide with the length and width of the absorbent article 10. As illustrated in FIG. 2, the absorbent article 10 can have a longitudinal axis 29 extending in the longitudinal direction 30 and a lateral axis 31 extending in the lateral direction 32. The longitudinal axis 29 and the lateral axis 31 can define a horizontal plane. The absorbent article 10 can also have a vertical axis 33 extending in a vertical direction 34. The vertical axis 33 is perpendicular to the horizontal plane defined by the longitudinal axis 29 and the lateral axis 31.

FIG. 2 illustrates the absorbent article 10 with certain portions cut-away for illustrating additional aspects of the absorbent article 10. An absorbent body 35 can be disposed between the backsheet 26 and the body facing liner 28. The absorbent body 35 can have longitudinal edges, 36 and 38, which, in an embodiment, can form portions of the longitudinal side edges, 18 and 20, respectively, of the absorbent article 10 and can have opposite end edges, 40 and 42, which, in an embodiment, can form portions of the waist edges, 22 and 24, respectively, of the absorbent article 10. In an embodiment, the absorbent body 35 can have a length and width that are the same as or less than the length and width of the absorbent article 10. The absorbent article 10 can also include a fluid acquisition layer 70 and a fluid transfer layer 66.

The absorbent article 10 can be configured to contain and/or absorb liquid, solid, and semi-solid body exudates discharged from the wearer. For example, containment flaps, 44 and 46, can be configured to provide a barrier to the lateral flow of body exudates. As illustrated in FIG. 2, each containment flap 44, 46 can include elastic members 48, 50. The elastic members 48, 50 can include one or more elastic strands (two are shown in FIG. 2) that are aligned substantially parallel to the longitudinal axis 29 of the absorbent article 10. The containment flaps 44, 46 are laterally spaced from one another, such that the containment flap 44 is on one side of the longitudinal axis 29 and the containment flap 46 is on an opposite side of the longitudinal axis 29. The containment flaps 44, 46 can be attached to the absorbent article 10 by being bonded to the body facing liner 28. The containment flaps, 44 and 46, can be located laterally inward from the longitudinal side edges, 18, 20 of the absorbent article 10, and can extend longitudinally along the entire length of absorbent article 10 or can extend partially along the length of the absorbent article 10.

To further enhance containment and/or absorption of body exudates, in some embodiments the absorbent article 10 can suitably include a rear waist elastic member 52, a front waist elastic member 54, and leg elastic members, 56 and 58, as are known to those skilled in the art. The waist elastic members, 52 and 54, can be attached to the backsheet 26 and/or the body facing liner 28 along the opposite waist edges, 24 and 22, and can extend over part or all of the waist edges, 24 and 22. In an embodiment shown in FIG. 2, the rear waist elastic member 52 is attached to the body facing liner 28 and the containment flaps 44, 46 and the front waist elastic member 54 is attached to the backsheet 26. The leg elastic members, 56 and 58, can be attached to the backsheet 26 and/or the body facing liner 28 along the opposite longitudinal side edges, 18 and 20, and positioned in the crotch region 16 of the absorbent article 10.

Additional details regarding each of these elements of the absorbent article 10 described herein can be found below and with reference to the FIGS. 1 through 8.

Backsheet:

The backsheet 26 and/or portions thereof can be breathable and/or liquid impermeable. The backsheet 26 and/or portions thereof can be elastic, stretchable, or non-stretchable. The backsheet 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the backsheet 26 can be constructed of a microporous polymeric film, such as polyethylene or polypropylene.

In an embodiment, the backsheet 26 can be a single layer of a liquid impermeable material. In an embodiment, the backsheet 26 can be suitably stretchable, and more suitably elastic, in at least the lateral or circumferential direction 32 of the absorbent article 10. In an embodiment, the backsheet 26 can be stretchable, and more suitably elastic, in both the lateral 32 and the longitudinal 30 directions. In an embodiment, the backsheet 26 can be a multi-layered laminate in which at least one of the layers is liquid impermeable. In an embodiment, the backsheet 26 can be a two layer construction, including an outer layer 60 material and an inner layer 62 material which can be bonded together such as by a laminate adhesive. Suitable laminate adhesives can be applied continuously or intermittently as beads, a spray, parallel swirls, or the like, but it is to be understood that the inner layer 62 can be bonded to the outer layer 60 by other bonding methods, including, but not limited to, ultrasonic bonds, thermal bonds, pressure bonds, or the like.

The outer layer 60 of the backsheet 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 60 of a backsheet 26 can be a 20 gsm spunbond polypropylene non-woven web. The outer layer 60 may also be constructed of the same materials from which the body facing liner 28 can be constructed as described herein.

The liquid impermeable inner layer 62 of the backsheet 26 (or the liquid impermeable backsheet 26 where the backsheet 26 is of a single-layer construction) can be either vapor permeable (i.e., “breathable”) or vapor impermeable. The liquid impermeable inner layer 62 (or the liquid impermeable backsheet 26 where the backsheet 26 is of a single-layer construction) may be manufactured from a thin plastic film, although other liquid impermeable materials may also be used. The liquid impermeable inner layer 62 (or the liquid impermeable backsheet 26 where the backsheet 26 is of a single-layer construction) can inhibit liquid body exudates from leaking out of the absorbent article 10 and wetting articles, such as bed sheets and clothing, as well as the wearer and caregiver. An example of a material for a liquid impermeable inner layer 62 (or the liquid impermeable backsheet 26 where the backsheet 26 is of a single-layer construction) can be a printed 19 gsm Berry Plastics XP-8695H film or equivalent commercially available from Berry Plastics Corporation, Evansville, Ind., U.S.A.

Where the backsheet 26 is of a single layer construction, it can be embossed and/or matte finished to provide a more cloth-like texture or appearance. The backsheet 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

Absorbent Body:

The absorbent body 35 can be suitably constructed to be generally compressible, conformable, pliable, non-irritating to the wearer's skin and capable of absorbing and retaining liquid body exudates. The absorbent body 35 can be manufactured in a wide variety of sizes and shapes (for example, rectangular, trapezoidal, T-shape, I-shape, hourglass shape, etc.) and from a wide variety of materials. The size and the absorbent capacity of the absorbent body 35 should be compatible with the size of the intended wearer (infants to adults) and the liquid loading imparted by the intended use of the absorbent article 10. The absorbent body 35 can have a length and width that can be less than or equal to the length and width of the absorbent article 10. The absorbent body 35 can have two surfaces such as a wearer facing surface 64 and a garment facing surface (not shown). Edges, such as longitudinal side edges, 36 and 38, and such as front and back end edges, 40 and 42, can connect the two surfaces, 64 and 66.

In an embodiment, the absorbent body 35 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 35 can be a matrix of cellulosic fluff and superabsorbent material. In an embodiment, the absorbent body 35 may be constructed of a single layer of materials, or in the alternative, may be constructed of two or more layers of materials.

Various types of wettable, hydrophilic fibers can be used in the absorbent body 35. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 35 can be free of superabsorbent material.

The absorbent body 35 can be superposed over the inner layer 62 of the backsheet 26, extending laterally between the leg elastic members, 56, 58, and can be bonded to the inner layer 62 of the backsheet 26, such as by being bonded thereto with adhesive. However, it is to be understood that the absorbent body 35 may be in contact with, and not bonded with, the backsheet 26 and remain within the scope of this disclosure. In an embodiment, the backsheet 26 can be composed of a single layer and the absorbent body 35 can be in contact with the singer layer of the backsheet 26. In an embodiment, a layer, such as but not limited to, a fluid transfer layer 66, can be positioned between the absorbent body 35 and the backsheet 26.

Fluid Transfer Layer:

In various embodiments an absorbent article 10 can be constructed without a fluid transfer layer 66. In various embodiments the absorbent article 10 can have a fluid transfer layer 66. In an embodiment, the fluid transfer layer 66 can be in contact with the absorbent body 35. In an embodiment, the fluid transfer layer 66 can be bonded to the absorbent body 35. Bonding of the fluid transfer layer 66 to the absorbent body 35 can occur via any means known to one of ordinary skill, such as, but not limited to, adhesives. In an embodiment, a fluid transfer layer 66 can be positioned between the body facing liner 28 and the absorbent body 35. In an embodiment, a fluid transfer layer 66 can completely encompass the absorbent body 35 and can be sealed to itself. In an embodiment a fluid transfer layer 66 may be composed of separate sheets of material which can be utilized to partially or fully encompass the absorbent body 35 and which can be sealed together using a sealing means such as, but not limited to, an ultrasonic bonder or other thermochemical bonding means or the use of an adhesive. In an embodiment, the fluid transfer layer 66 can be in contact with and/or bonded with the wearer facing surface 64 of the absorbent body 35.

The fluid transfer layer 66 can be pliable, less hydrophilic than the absorbent body 35, and sufficiently porous to thereby permit liquid body exudates to penetrate through the fluid transfer layer 66 to reach the absorbent body 35. In an embodiment, the fluid transfer layer 66 can have sufficient structural integrity to withstand wetting thereof and of the absorbent body 35. In an embodiment, the fluid transfer layer 66 can be constructed from a single layer of material or it may be a laminate constructed from two or more layers of material. In an embodiment, the fluid transfer layer 66 can include, but is not limited to, natural and synthetic fibers such as, but not limited to, polyester, polypropylene, acetate, nylon, polymeric s materials, cellulosic materials and combinations thereof. In an embodiment, a fluid transfer layer 66 can include spunbond and/or meltblown materials. In an embodiment, the fluid transfer layer 66 can be a laminate of a meltblown nonwoven material having fine fibers laminated to at least one spunbond nonwoven material layer having coarse fibers. In such an embodiment, the fluid transfer layer 66 can be a spunbond-meltblown (“SM”) material, while in other embodiments, the fluid transfer layer 66 can be a spunbond-meltblown-spunbond (“SMS”) material. In various embodiments, the fluid transfer layer 66 can be hydrophilic. In various embodiments, the fluid transfer layer 66 can be hydrophobic and can be treated in any manner known in the art to be made hydrophilic. In an embodiment, the fluid transfer layer 66 can have a longitudinal length the same as, greater than, or less than the longitudinal length of the absorbent body 35.

Fluid Acquisition Layer:

In various embodiments the absorbent article 10 can have a fluid acquisition layer 70. The acquisition layer 70 can help decelerate and diffuse surges or gushes of liquid body exudates penetrating the body facing liner 28. In an embodiment, the acquisition layer 70 can be positioned between the body facing liner 28 and the backsheet 26. In an embodiment, the acquisition layer 70 can be positioned between the body facing liner 28 and the absorbent body 35 to take in and distribute body exudates for absorption by the absorbent body 35. In an embodiment, the acquisition layer 70 can be positioned between the body facing liner 28 and a fluid transfer layer 66 if a fluid transfer layer 66 is present.

In an embodiment, the acquisition layer 70 can be in contact with and/or bonded with the body facing liner 28. In an embodiment in which the acquisition layer 70 is bonded with the body facing liner 28, bonding of the acquisition layer 70 to the body facing liner 28 can occur through the use of an adhesive and/or point fusion bonding, but is not limited to such methods of bonding. For example, the body facing liner 28 could be bonded to the acquisition layer 70 by hydroentangling the body facing liner 28 with the acquisition layer 70. The point fusion bonding can be selected from, but is not limited to, ultrasonic bonding, pressure bonding, thermal bonding, and combinations thereof. In an embodiment, the point fusion bonding can be provided in any pattern as deemed suitable. As an example, the body facing liner 28 can be bonded to the acquisition layer 70 at a range of 1-90%. The percentage of bonding between the body facing liner 28 and the acquisition layer 70 can be measured by calculating the area of bonded material between the body facing liner 28 and the acquisition layer 70 and dividing by the area of overlap between the body facing liner 28 and the acquisition layer 70 as viewed from the vertical direction 34 perpendicular to both the longitudinal and lateral directions 30, 32, as in a dimension that is perpendicular to the plane of the body facing liner 28 when the body facing liner 28 is laid flat.

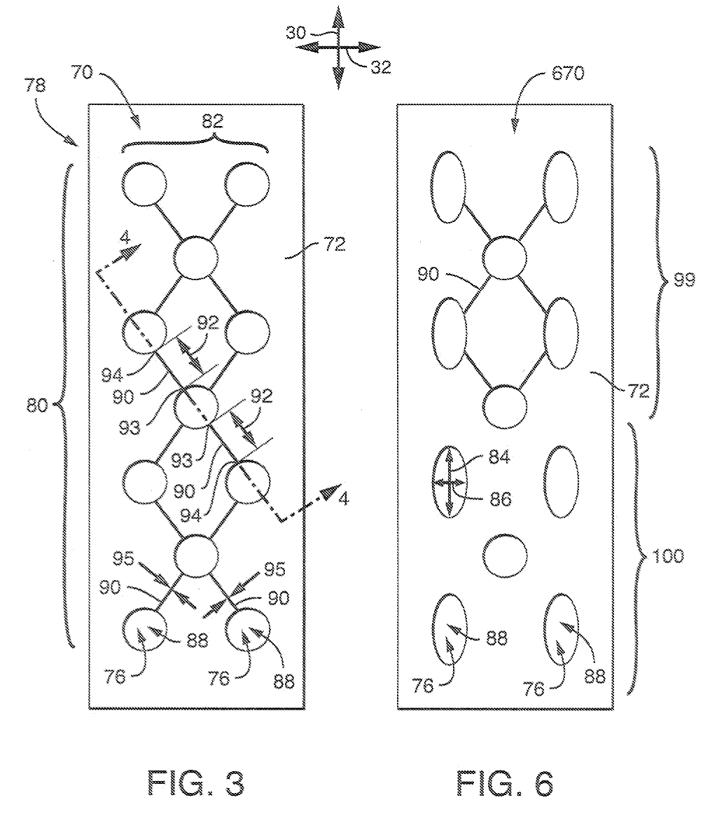

The acquisition layer 70 can be rectangular in shape, such as shown in FIGS. 3, 6, and 7, hourglass in shape, or can be any other shape. The acquisition layer 70 may have any longitudinal length dimension as deemed suitable. For example, the acquisition layer 70 can have a longitudinal length shorter than, the same as, or longer than the longitudinal length of the absorbent body 35. In an embodiment, the acquisition layer 70 can have any length such that the acquisition layer 70 can be coterminous with the waist edges, 22 and 24, of the absorbent article 10. In an embodiment, the longitudinal length of the acquisition layer 70 can be the same as the longitudinal length of the absorbent body 35. In such an embodiment, the midpoint of the longitudinal length of the acquisition layer 70 can substantially align with the midpoint of the longitudinal length of the absorbent body 35.

In an embodiment, the longitudinal length of the acquisition layer 70 can be shorter than the longitudinal length of the absorbent body 35. In such an embodiment, the acquisition layer 70 may be positioned at any desired location along the longitudinal length of the absorbent body 35. As an example of such an embodiment, the absorbent article 10 may contain a target area where repeated surges of bodily exudates typically occur in the absorbent article 10. The particular location of a target area can vary depending on the type of bodily exudate and/or on the age and gender of the wearer of the absorbent article 10. For example, a target area of the absorbent article 10 can vary based on urine or fecal matter. With respect to urination, males tend to urinate further toward the front waist region 12 of the absorbent article 10 and the target area may be phased forward within the absorbent article 10. For example, the target area for a male wearer may be positioned about 2¾″ forward of the longitudinal midpoint of the absorbent body 35 and may have a length of about ±3″ and a width of about ±2″. The female target area with respect to urination can be located closer to the center of the crotch region 16 of the absorbent article 10. For example, the target area for a female wearer may be positioned about 1″ forward of the longitudinal midpoint of the absorbent body 35 and may have a length of about ±3″ and a width of about ±2″. Thus, the relative longitudinal placement of the acquisition layer 70 within the absorbent article 10 can be selected to best correspond with the target area of either or both categories of wearers and can depend on what type of bodily exudate is specifically being targeted to control.

In an embodiment, the absorbent article 10 may contain a target area centered within the crotch region 16 of the absorbent article 10 with the premise that the absorbent article 10 would be worn by a female wearer. The acquisition layer 70, therefore, may be positioned along the longitudinal length of the absorbent article 10 such that the acquisition layer 70 can be substantially aligned with the target area of the absorbent article 10 intended for a female wearer. Alternatively, the absorbent article 10 may contain a target area positioned between the crotch region 16 and the front waist region 12 of the absorbent article 10 with the premise that the absorbent article 10 would be worn by a male wearer. The acquisition layer 70, therefore, may be positioned along the longitudinal length of the absorbent article 10 such that the acquisition layer 70 can be substantially aligned with the target area of the absorbent article 10 intended for a male wearer. In an embodiment, the acquisition layer 70 can have a size dimension that is the same size dimension as the target area of the absorbent article 10 or a size dimension greater than the size dimension of the target area of the absorbent article 10. In an embodiment, the acquisition layer 70 can be in contact with and/or bonded with the body facing liner 28 at least partially in the target area of the absorbent article 10.

In various embodiments, the acquisition layer 70 can have a longitudinal length shorter than, the same as, or longer than the longitudinal length of the absorbent body 35. In an embodiment in which the absorbent article 10 is a diaper, the acquisition layer 70 may have a longitudinal length from about 120, 130, 140, 150, 160, 170, or 180 mm to about 200, 210, 220, 225, 240, 260, 280, 300, 310 or 320 mm. In such an embodiment, the acquisition layer 70 may be shorter in longitudinal length than the longitudinal length of the absorbent body 35 and may be phased from the front end edge 40 or the back end edge 42 of the absorbent body 35 a distance of from about 15, 20, or 25 mm to about 30, 35 or 40 mm. In an embodiment in which the absorbent article 10 may be a training pant or youth pant, the acquisition layer 70 may have a longitudinal length from about 120, 130, 140, 150, 200, 210, 220, 230, 240 or 250 mm to about 260, 270, 280, 290, 300, 340, 360, 400, 410, 420, 440, 450, 460, 480, 500, 510 or 520 mm. In such an embodiment, the acquisition layer 70 may have a longitudinal length shorter than the longitudinal length of the absorbent body 35 and may be phased a distance of from about 25, 30, 35 or 40 mm to about 45, 50, 55, 60, 65, 70, 75, 80 or 85 mm from the front end edge 40 of the absorbent body 35. In an embodiment in which the absorbent article 10 is an adult incontinence garment, the acquisition layer 70 may have a longitudinal length from about 200, 210, 220, 230, 240, or 250 mm to about 260, 270, 280, 290, 300, 320, 340, 360, 380, 400, 410, 415, 425, or 450 mm. In such an embodiment, the acquisition layer 70 may have a longitudinal length shorter than the longitudinal length of the absorbent body 35 and the acquisition layer 70 may be phased a distance of from about 20, 25, 30 or 35 mm to about 40, 45, 50, 55, 60, 65, 70 or 75 mm from the front end edge 40 of the absorbent body 35.

The acquisition layer 70 may have any width as desired. The width of the acquisition layer 70 may vary dependent upon the size and shape of the absorbent article 10 within which the acquisition layer 70 will be placed. The acquisition layer 70 can have a width smaller than, the same as, or larger than the width of the absorbent body 35. Within the crotch region 16 of the absorbent article 10, the acquisition layer 70 can have a width smaller than, the same as, or larger than the width of the absorbent body 35.

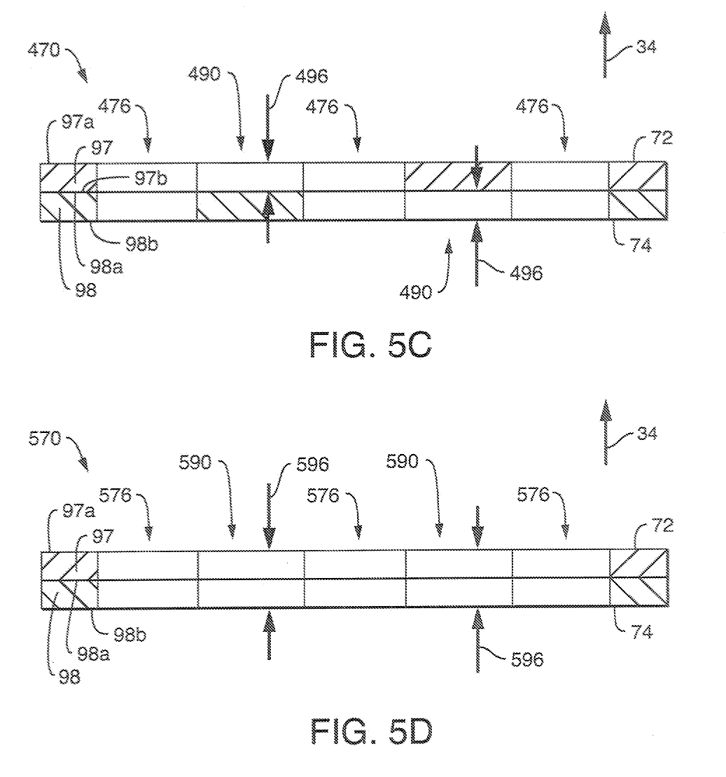

The acquisition layer 70 can have a body facing surface 72 and a garment facing surface 74. The acquisition layer 70 can have at least one aperture 76. In some embodiments, the acquisition layer 70 can have a plurality of apertures 76 (only two of the apertures 76 being labeled in FIGS. 3 and 6 for clarity purposes). In some embodiments, the apertures 76 can extend from the body facing surface 72 of the acquisition layer 70 to the garment facing surface 74 of the acquisition layer 70. The plurality of apertures 76 can be in a pattern 78 that form a plurality of rows 80 and a plurality of columns 82, as labeled in FIG. 3. In some embodiments, the plurality of apertures 72 can be uniformly distributed throughout the length and/or the width of the acquisition layer 70, while in other embodiments, the plurality of aperture 72 can be non-uniformly distributed throughout the length and/or the width of the acquisition layer 70.

The apertures 76 in the acquisition layer 70 can be of various shapes and sizes, and some apertures 76 can be different from the size and/or shape of other apertures 76 in a pattern 78. For example, the apertures 76 can be circular in shape, as shown in the exemplary embodiment in FIG. 3. However, some apertures 76 can alternatively be elliptical in shape (such as shown in FIG. 6) or be of any other suitable shape, including shapes other than circular and elliptical, including, but not limited to, regular and irregular polygons (regular and irregular triangles, regular and irregular rectangles, regular and irregular pentagons, etc. . . . ), and irregular shapes. As shown in FIG. 3, the acquisition layer 70 can have a pattern 78 of apertures 76 in which all of the apertures 76 are of substantially the same shape and size. As shown in FIG. 6, the acquisition layer 70 can have a pattern 78 of apertures 76 in which not all of the apertures 76 are of the same shape.

The size of the apertures 76 in the acquisition layer 70 can also vary. For example, in an exemplary embodiment, an aperture 76 that is circular in shape can have a diameter in the range of 1.0 mm-100.0 mm, preferably in the range of 4.0 mm-50.0 mm, and more preferably in the range of 6.0 mm-20.0 mm. In another exemplary embodiment, an aperture 76 that is elliptical in shape, such as that shown in FIG. 6, the major axis 84 of the aperture 76 can range from 1.0 mm-100.0 mm, preferably in the range of 4.0 mm-50.0 mm, and more preferably in the range of 6.0 mm-20.0 mm. In such an embodiment, the minor axis 86 of an aperture 76 that is elliptical in shape can range from 0.5 mm to 100.0 mm, preferably in the range of 0.5 mm-45.0 mm, and more preferably in the range of 3.0 mm-15.0 mm. Additionally, although the major axis 84 of the elliptical shaped apertures 76 can be aligned with the longitudinal direction 30 as shown in FIG. 6, the major axis 84 could be designed to be parallel with the lateral direction 32, or form an acute angle with respect to the longitudinal direction 30.

As briefly mentioned above, the pattern 78 of apertures 76 in the acquisition layer 70 can form a plurality of rows 80 and a plurality of columns 82, as labeled in FIG. 3. The rows 80 of apertures 76 can extend in a direction parallel to the lateral axis 31 and can be offset from one another in a direction parallel to the longitudinal axis 29. The columns 82 of apertures 76 can extend in a direction parallel to the longitudinal axis 29 and can be offset from one another in a direction parallel to the lateral axis 31. Of course, it is contemplated that the rows 80 and columns 82 of apertures 76 are not limited to such orientations. In an embodiment, the number of rows 80 of apertures 76 can be selected from the range of 1-50, preferably from the range of 4-30, and more preferably from the range of 6-20. In one embodiment, the number of columns 82 of apertures 76 can be selected from the range of 1-25, preferably from the range of 2-20, and more preferably from the range of 3-15. In the exemplary embodiment depicted in FIG. 3, the acquisition layer 70 can include seven rows 80 of apertures 76 and three columns 82 of apertures 76. The pattern 78 of apertures 76, including the amount and the positioning of the rows 80 and columns 82 of the apertures 76, can correspond to features in the body facing liner 28, as will be discussed in further detail below. The pattern 78 of apertures 76 depicted in the exemplary embodiment of FIG. 3 has some rows 80 that include two apertures 76 and some rows 80 that include one aperture 76, and some columns 82 that include four apertures 76 and one column 82 that includes three apertures 76. The rows 80 and the columns 82 can be configured such that there is phasing between adjacent rows 80 and/or adjacent columns 82 of the pattern 78 of apertures 76 such that adjacent rows 80 do not have apertures 76 aligned in the longitudinal direction 30 and adjacent columns 82 do not have apertures 76 aligned in the lateral direction 32. Phasing of the rows 80 and/or columns 82 of the apertures 76 can provide the advantages of more dense patterns 78 of apertures 76, or more apertures 76 per unit area of acquisition layer 70 to possibly provide increased intake and/or distribution results. Additionally, the phasing of the rows 80 and/or columns 82 of the apertures 76 can provide for higher tensile strengths of the acquisition layer 70 in both the longitudinal 30 and lateral 32 directions compared to similar patterns 76 that do not have phased rows 80 and/or columns 82.

Each aperture 76 in the acquisition layer 70 provides an open area 88 in the acquisition layer 70 (only two of the open areas 88 being labeled in FIGS. 3 and 6 for clarity purposes) that can assist fluid and/or particulate matter exudates to transfer through the acquisition layer 70. The sum of the open areas 88 for each of the plurality of apertures 76 provides a total open area for the acquisition layer 70. In exemplary embodiments, the total open area of the acquisition layer 70 can range from 1% to 70% of the total area of the acquisition layer 70, more preferably can range from 5% to 45% of the total area of the acquisition layer 70, and even more preferably can range from 10% to 40% of the total area of the acquisition layer 70.

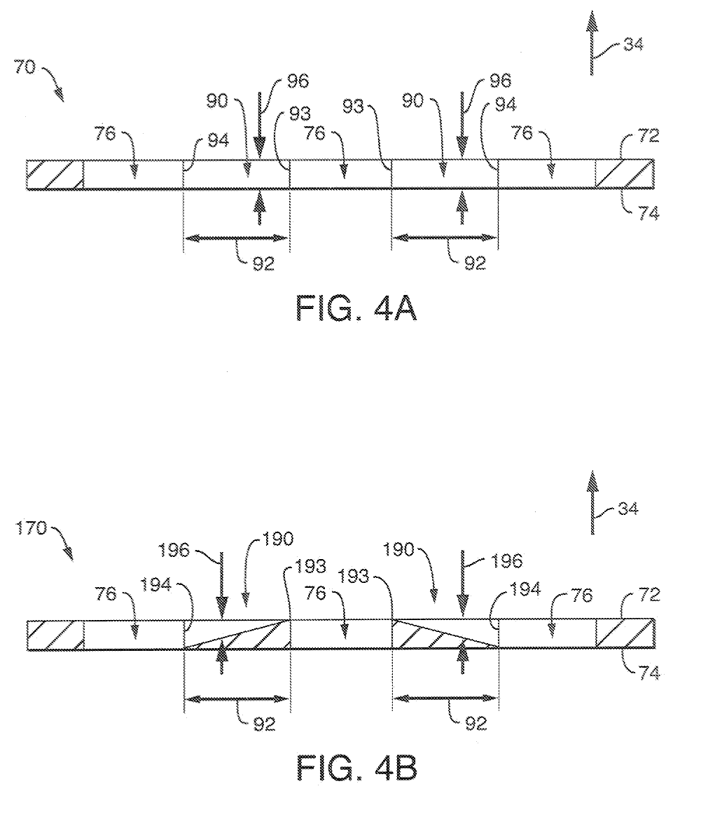

The fluid acquisition layer 70 can also include a channel 90, or a plurality of channels 90, as shown in FIGS. 3 and 4A. The acquisition layer 70 illustrated in FIG. 3 includes twelve channels 90, (only four being labeled for clarity purposes). FIG. 4A provides a cross-sectional view taken along line 4-4 from FIG. 3 and illustrates three apertures 76 and two channels 90 that extend between the apertures 76. In some embodiments, at least one aperture 76 can be connected to at least two channels 90. As shown in FIG. 3, some apertures 76 can be connected to four channels 90. It is contemplated that in some embodiments, apertures 76 can be connected to more than four channels 90.

As shown in FIGS. 3 and 4A, each channel 90 can include a length 92 that is measured in a horizontal plane parallel to the absorbent article 10 when the article 10 is in the laid flat condition, such as shown in FIG. 2, the horizontal plane including the longitudinal and lateral axes 30, 32, respectively. The length 92 of a channel 90 is defined between a proximal end 93 and a distal end 94 as measured in the horizontal plane discussed above. In some embodiments, the proximal end 93 of a channel 90 can be connected to an aperture 76, such as shown in FIG. 4A. In some embodiments, the distal end 94 of the same channel 90 can be connected to a different aperture 76 such that the length 92 of the channel 90 extends between, or connects, two apertures 76 of the plurality of apertures 76 in the acquisition layer 70. In some embodiments, a majority of the channels 90 in the acquisition layer 70 can include a proximal end 93 that is connected to at least one aperture 76 and a distal end 94 that is connected to a different aperture 76. In some embodiments, such as that shown in FIGS. 3 and 6, all of the channels 90 in the acquisition layer 70 can include a proximal end 93 that is connected to at least one aperture 76 and a distal end 94 that is connected to a different aperture 76. The length 92 of a channel 90 in the acquisition layer 70 can vary, however, in exemplary embodiments, the length 92 of a channel 90 can range between 1-100 mm, preferably between 5-30 mm, and more preferably between 10-20 mm. In some embodiments including more than one channel 90, the length 92 of each channel 90 can be the same for the fluid acquisition layer 70. Although, it is to be noted that the length 92 can vary among different channels 90 in the same fluid acquisition layer 70.

Each channel 90 can also include a thickness 95 measured in the horizontal plane parallel to the absorbent article 10 when the article 10 is in the laid flat condition. Regardless of the orientation of the channel 90 in the horizontal plane, the length 92 of the channel 90 will be greater than or equal to the thickness 95 of the channel 90. The thickness 95 of a channel 90 in the acquisition layer 70 can vary, however, in exemplary embodiments, the thickness 95 of a channel 90 can range between 1-30 mm, preferably between 3-15 mm, and more preferably between 4-8 mm. In some embodiments including more than one channel 90, the thickness 95 of each channel 90 can be the same for the fluid acquisition layer 70. However, it is to be noted that the thickness 95 can vary among different channels 90 in the same fluid acquisition layer 70.

Each channel 90 can also have a depth 96 that is defined in a vertical direction 34 that is parallel to the vertical axis 33. The depth 96 of the channels 90 can extend from the body facing surface 72 of the acquisition layer 70 to the garment facing surface 74 of the acquisition layer 70. As shown in the exemplary embodiment in FIG. 4A, the depth 96 of the channels 90 can be the same along the length 92 of the channels 90. However, in another embodiment of a fluid acquisition layer 170 such as that depicted in FIG. 4B, the depth 196 of a channel 190 can vary along the length 192 of the channel 190. In some embodiments, at the distal end 194 of the channel 190 the depth 196 can extend from the body facing surface 72 of the acquisition layer 170 to the garment facing surface 74 of the acquisition layer 170. As illustrated in FIG. 4B, the depth 196 at the proximal end 193 of the channel 190 can be less than the depth 196 at the distal end 194 of the channel 190. Of course, it is contemplated that the depth 196 at the proximal end 193 of the channel 190 can be greater than the depth 196 at the distal end 194 of the channel 190 in other embodiments, and as such, such a configuration is within the spirit and scope of this disclosure. In some embodiments including more than one channel 90, the depth 96 of each channel 90 can be the same for the fluid acquisition layer 70. It is to be noted that the depth 96 can vary among different channels 90 in the same fluid acquisition layer 70.

The channels 90, 190 in the acquisition layers 70, 170 depicted in the embodiments shown in FIGS. 2-4B provide advantages in the distribution of the exudates in an absorbent article 10. For example, when exudates transfer through the body facing liner 28 towards the absorbent body 35, the exudates can contact the body facing surface 72 of the acquisition layer 70, 170. The apertures 76 can provide open area 88 in the acquisition layer 70, 170 to accept the exudates and transfer the exudates to other layers of the absorbent article 10, such as a fluid transfer layer 66 and/or the absorbent body 35. If exudates are concentrated in any particular area of the acquisition layer 70, 170, channels 90, 190 in the acquisition layer 70, 170 can provide reduced resistance in the acquisition layer 70, 170 for the exudates to flow to spread throughout the acquisition layer 70, 170. Such a configuration is believed to increase the effectiveness of distribution of exudates in the acquisition layer 70, 170, especially with semi-solid fecal matter, which may otherwise have difficulty penetrating and spreading throughout the acquisition layer 70, 170. A more effective distribution of the of exudates in the acquisition layer 70, 170 can provide an increase in the effectiveness of the absorbent body 35, which in turn can reduce the potential for skin irritation of the wearer by reducing the amount of exudates that may return to the body facing liner 28. Also, the increase in distribution of the exudates throughout the acquisition layer 70, 170, and in turn an increase in the effectiveness of the absorbent body 35, can assist in reducing the likelihood that the gasketing of the absorbent article 10 may be compromised.

In exemplary embodiments, the acquisition layer 70, 170 can include woven materials; fibrous nonwovens such as spunbond webs, meltblown webs and carded webs such as airlaid webs, bonded carded webs, and coform materials; binder and calendar bonded webs; foams, including open-cell foams;

and scrim materials. The acquisition layer 70, 170 can include various types of fibers such as natural fibers; cellulosic fibers; synthetic fibers composed of cellulose or cellulose derivatives, such as rayon fibers; inorganic fibers composed of an inherently wettable material, such as glass fibers; and synthetic fibers made from inherently wettable thermoplastic polymers, such as particular polyester or polyamide fibers, or nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. In some embodiments, the acquisition layer 70, 170 can be formed from a material that is substantially hydrophobic, such as a nonwoven web composed of polypropylene, polyethylene, polyester, and the like, and combinations thereof. In some embodiments, the acquisition layer 70, 170 can include superabsorbent material. In some embodiments, the acquisition layer 70, 170 can include materials having a basis weight ranging from about 10 gsm to about 300 gsm.

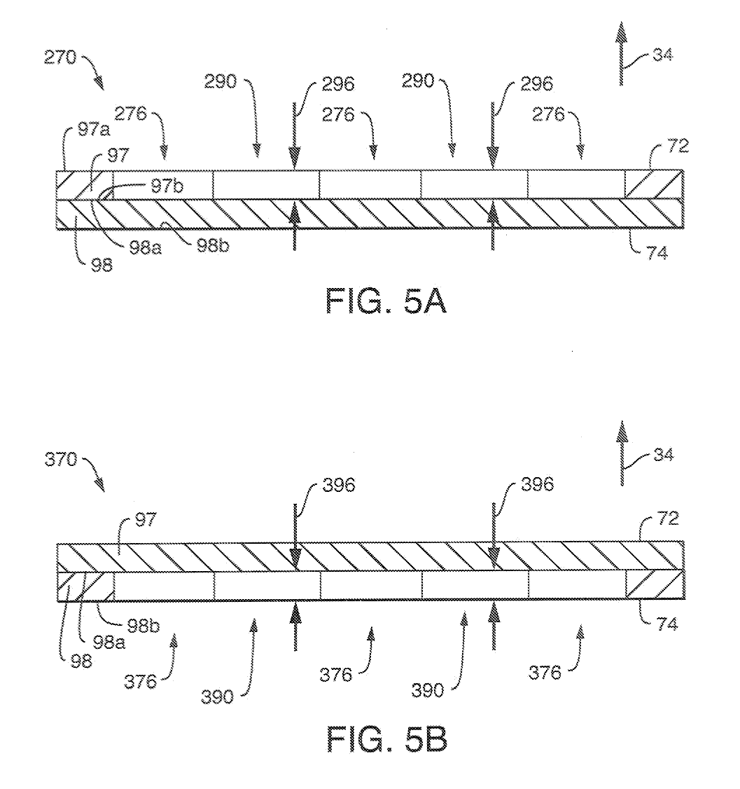

Turning now to FIGS. 5A-5D, alternative embodiments and configurations of fluid acquisition layers 270, 370, 470, 570 that include more than one layer will now be discussed. FIGS. 5A-5D provide a cross-sectional illustration similar to the cross-sectional illustrations in FIGS. 4A and 4B. FIGS. 5A-5D each depict fluid acquisition layers 270, 370, 470, 570 that can include a first layer 97 and a second layer 98. A top surface 97a of the first layer 97 can provide the body facing surface 72 of the acquisition layer 270, 370, 470, 570. A bottom surface 98b of the second layer 98 can provide the garment facing surface 74 of the acquisition layer 270, 370, 470, 570. The bottom surface 97b of the first layer 97 can be directly bonded to the top surface 98a of the second layer 98 by adhesives, pressure bonding, ultrasonic bonding, and other suitable methods known by those having ordinary skill in the art.

In the embodiment depicted in FIG. 5A, the first layer 97 can include apertures 276 that extend completely through the first layer 97, or from the top surface 97a to the bottom surface 97b of the first layer 97. The first layer 97 can also include channels 290. The channels 290 can extend between the apertures 276, as described above with respect to FIGS. 3 and 4A. In the embodiment depicted in FIG. 5A, the channels 290 can include a depth 296 that extends from the top surface 97a of the first layer 97 to the bottom surface 97b of the first layer 97. The second layer 98 can be free from apertures 276 and channels 290.

FIG. 5B depicts an alternative configuration of a multilayered acquisition layer 370. In the embodiment depicted in FIG. 5B, the second layer 98 can include apertures 376 and channels 390 extending between the apertures 376. The apertures 376 can extend completely through the second layer 98, or in other words, from the top surface 98a to the bottom surface 98b of the second layer 98. The channels 390 can include a depth 396 extending from the top surface 98a of the second layer 98 to the bottom surface 98b of the second layer 98 of the acquisition layer 370.

In the embodiments depicted in FIGS. 5A and 5B, the acquisition layers 270, 370 each include a two-layered structure, with one layer that is free from apertures 276, 376, respectively, and channels 290, 390, respectively. As a result, this construction can provide an increase in strength to the acquisition layer 270, 370 by having at least the first layer 97 or the second layer 98 free from such features. Increasing the strength of the acquisition layer 270, 370 in this nature can provide enhanced manufacturability of the acquisition layer 270, 370 when in roll form, while still providing at least some of the benefits noted above with respect to increased effectiveness in distribution of exudates.

FIG. 5C provides yet another alternative embodiment of an acquisition layer 470 that includes a multilayered structure. In FIG. 5C, the acquisition layer 470 can include apertures 476 that extend from the top surface 97a of the first layer 97 to the bottom surface 98b of the second layer 98, or in other words, extend from the body facing surface 72 of the acquisition layer 470 to the garment facing surface 74 of the acquisition layer 470. The first layer 97 can include at least one channel 490 and the second layer 98 can also include at least one channel 490. The channels 490 can extend between the adjacent apertures 476 in the acquisition layer 470, as discussed above. The channel 490 in the first layer 97 can include a depth 496 extending from the top surface 97a of the first layer 97 to the bottom surface 97b of the first layer 97. The channel 490 in the second layer 98 can include a depth 496 extending from the top surface 98a of the second layer 98 to the bottom surface 98b of the second layer 98. Configuring the acquisition layer 470 to include some channels 490 in the first layer 97 and some channels 490 in the second layer 98 can provide an alternative configuration to that as shown in FIGS. 5A and 5B to help improve the strength of the acquisition layer 470, while still seeking enhanced distribution of exudates in the acquisition layer 470.

FIG. 5D provides yet another alternative embodiment of an acquisition layer 570 that includes more than one layer. In FIG. 5D, the acquisition layer 570 can include apertures 576 that extend from the top surface 97a of the first layer 97 to the bottom surface 98b of the second layer 98, or in other words, extend from the body facing surface 72 of the acquisition layer 570 to the garment facing surface 74 of the acquisition layer 570. The acquisition layer 570 can also include channels 590 extending between the apertures 576. The channels 590 can include a depth 596 extending from the top surface 97a of the first layer 97 to the bottom surface 98b of the second layer 98, or stated differently, from the body facing surface 72 of the acquisition layer 570 to the garment facing surface 74 of the acquisition layer 570.

In the exemplary embodiments depicted in FIGS. 5A-5D and discussed above, the first layer 97 and the second layer 98 of the acquisition layer 270 can be composed of the same material, or can be composed of different materials. In some embodiments, the first layer 97 and/or the second layer 98 can be composed of, but are not limited to, the following materials: fibrous nonwovens such as spunbond webs, meltblown webs and carded webs such as airlaid webs, bonded carded webs, and coform materials; binder and calendar bonded webs; polymer films; nonwoven/polymer film laminates; foams, including open-cell foams; and scrim materials. Various types of wettable, hydrophilic fibers can be used in the first layer 97 and/or the second layer 98. Examples of suitable fibers include, but are not limited to: natural fibers; cellulosic fibers; synthetic fibers composed of cellulose or cellulose derivatives, such as rayon fibers; inorganic fibers composed of an inherently wettable material, such as glass fibers; and synthetic fibers made from inherently wettable thermoplastic polymers, such as particular polyester or polyamide fibers, or nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. In some embodiments, the first layer 97 and/or the second layer 98 can be formed from a material that is substantially hydrophobic, such as a nonwoven web composed of polypropylene, polyethylene, polyester, and the like, and combinations thereof. In some embodiments, the first layer 97 and/or the second layer 98 can include superabsorbent material.

Additionally, the first layer 97 and the second layer 98 can have the same or different basis weights. In some embodiments, the first layer 97 and/or the second layer 98 can include materials having a basis weight ranging from about 10 gsm to about 300 gsm. Furthermore, the first layer 97 and the second layer 98 can include materials having the same or different densities, and the same or different porosities.

Various patterns of channels can exist in the acquisition layer. For example, FIG. 6 provides another exemplary embodiment of an acquisition layer 670 where channels 90 only extend between some of the apertures 76 in the acquisition layer 670 (only one channel 90 being labeled in FIG. 6 for clarity purposes). Additionally and/or alternatively, the channels 90 can be located in a channeled region 99 of the acquisition layer 670, and the acquisition layer 670 can include a non-channeled region 100 that can be free from channels 90. The channeled region 99 can be configured to correspond to a portion of the absorbent article 10 more likely to receive exudates in general, or particular forms of exudates. For example, the acquisition layer 670 depicted in FIG. 6 could be configured within an absorbent article 10 such that the channeled region 99 is located near the rear waist region 14 and/or the crotch region 16 which is more likely to be insulted with semi-solid fecal material. Alternatively, the acquisition layer 670 could be configured within an absorbent article 10 such that the channeled region 99 is located near the crotch region 16 and/or the front waist region 12. It is contemplated that this and similar configurations of channels 90 in the acquisition layer 670 as shown in FIG. 6 could be applied to other embodiments of the acquisition layers described above.

The apertures 76 and channels 90 can be formed in the acquisition layer 70 using various manufacturing techniques. For example, a pattern 78 of apertures 76 and channels 90 can be cut into the acquisition layer 70 by a rotary die (not shown), a laser cutter (not shown), a water cutter (not shown), or a punch press (not shown). The creation of the apertures 76 and channels 90 can be completed off the machine line forming absorbent articles 10, or can be completed in-line with the machine line forming absorbent articles 10. Advantageously, creating the apertures 76 in the acquisition layer 70 off-line allows the cutting to be completed at various speeds, including speeds that may be slower than the machine-line forming the absorbent articles 10, which may allow more precise cutting of the apertures 76. Furthermore, the production of the apertures 76 and channels 90 can be completed in the same process steps or in separate steps.

Body Facing Liner:

The body facing liner 28 of the absorbent article 10 can overlay the absorbent body 35 and the backsheet 26 and can isolate the wearer's skin from liquid waste retained by the absorbent body 35. In various embodiments, a fluid transfer layer 66 can be positioned between the body facing liner 28 and the absorbent body 35. In various embodiments, an acquisition layer 70 can be positioned between the body facing liner 28 and the absorbent body 35 or a fluid transfer layer 72, if present. In various embodiments, the body facing liner 28 can be bonded to the acquisition layer 70, or to the fluid transfer layer 66 if no acquisition layer 70 is present, via adhesive and/or by a point fusion bonding. The point fusion bonding may be selected from ultrasonic, thermal, pressure bonding, and combinations thereof.

In an embodiment, the body facing liner 28 can extend beyond the absorbent body 35 and/or a fluid transfer layer 66, and/or an acquisition layer 70 to overlay a portion of the backsheet 26 and can be bonded thereto by any method deemed suitable, such as, for example, by being bonded thereto by adhesive, to substantially enclose the absorbent body 35 between the backsheet 26 and the body facing liner 28. The body facing liner 28 may be narrower than the backsheet 26, but it is to be understood that the body facing liner 28 and the backsheet 26 may be of the same dimensions. It is also contemplated that the body facing liner 28 may not extend beyond the absorbent body 35 and/or may not be secured to the backsheet 26. It is further contemplated that the body facing liner 28 may be composed of more than one segment of material. The body facing liner 28 can be of different shapes, including rectangular, hourglass, or any other shape. The body facing liner 28 can be suitably compliant, soft feeling, and non-irritating to the wearer's skin and can be the same as or less hydrophilic than the absorbent body 35 to permit body exudates to readily penetrate through to the absorbent body 35 and provide a relatively dry surface to the wearer.

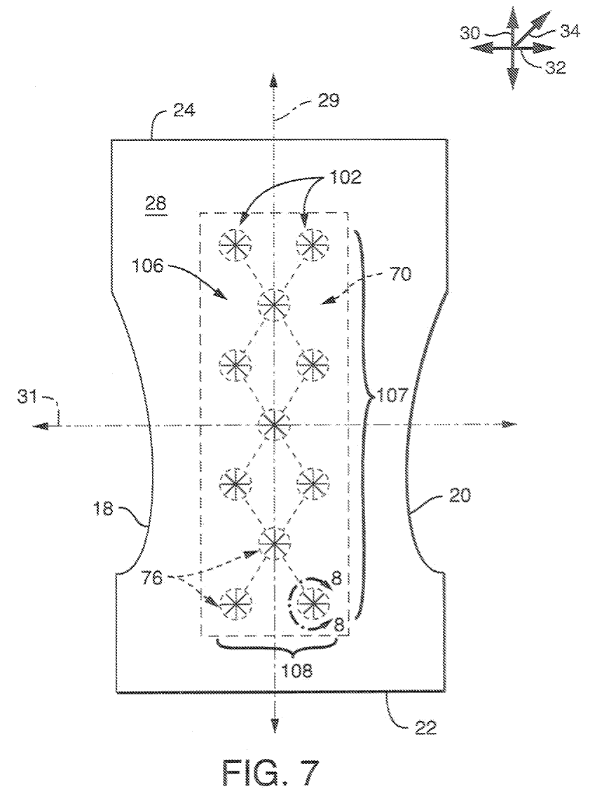

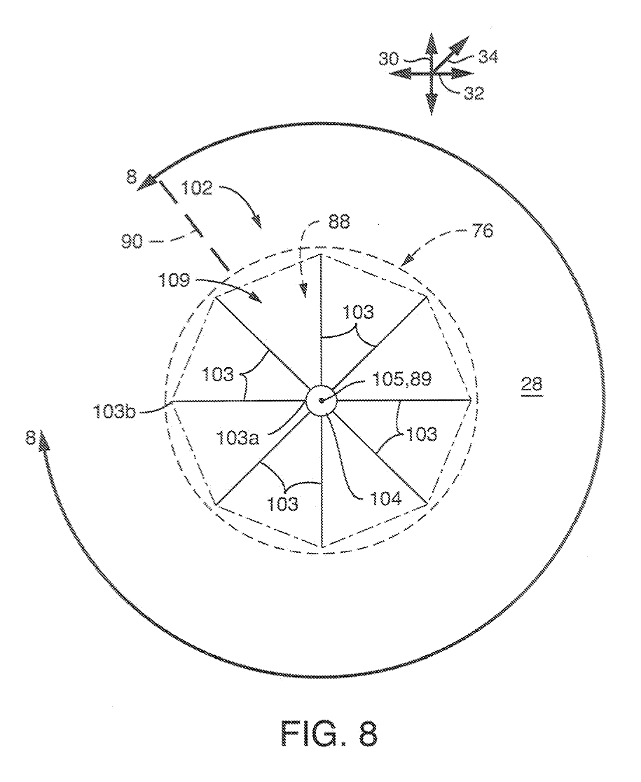

As shown in FIGS. 2, 7, and 8, the body facing liner 28 can include at least one intersecting slit formation 102. In some embodiments, the body facing liner 28 can include a plurality of intersecting slit formations 102 (only two intersecting slit formations 102 are labeled in FIGS. 2 and 7 for clarity purposes). The various characteristics of the intersecting slit formations 102 are described herein when the absorbent article 10 is in a stretched, laid flat configuration, such as that shown in FIG. 2. As shown in the detailed view of FIG. 8 depicting one exemplary embodiment of an intersecting slit formation 102 from the body facing liner 28, the intersecting slit formation 102 can include at least two intersecting slits 103 and an aperture 104. In a preferred embodiment, the intersecting slits 103 of the intersecting slit formation 102 can extend completely through a depth of the body facing liner 28, however, in other embodiments, some slits 103 of the intersecting slit formation 102 need not extend completely through the body facing liner 28. The intersecting slit formation 102 shown in FIG. 8 includes eight intersecting slits 103. It is contemplated that a body facing liner 28 could have an intersecting slit formation 102 with a specified amount of intersecting slits 103 selected from the range of 2-20 intersecting slits 103, more preferably from the range of 3-15 intersecting slits 103, and yet more preferably from the range of 5-8 intersecting slits 103. The intersecting slits 103 can intersect at a common intersection point 105, which can be within the aperture 104, if one is present in the intersecting slit formation 102.

The intersecting slits 103 are shown as linear segments, however, the intersecting slits 103 could be arcuate, sinusoidal, or in any other form or shape. An intersecting slit 103 can include a proximal end 103a and a distal end 103b, as labeled on only one of the slits 103 in FIG. 8 for clarity. A linear distance between the proximal end 103a and the distal end 103b can define a length of an intersecting slit 103. The intersecting slits 103 of the intersecting slit formation 102 can each be of the same length as depicted in FIG. 8, however, the intersecting slits 103 of the intersecting slit formation 102 can be of different lengths in comparison to one another. An intersecting slit 103 can be of a specified length selected from a range, including, but not limited to, 2-100 mm, more preferably 2-25 mm, and even more preferably, 3-15 mm. Additionally, the thickness of an exemplary intersecting slit 103 can be selected from the range of 0.02-5.00 mm, more preferably from the range of 0.05-2.00 mm, and even more preferably from the range of 0.10-1.50 mm. It can be appreciated, however, that the specified length and thickness of a slit 103 can deviate from the preferred ranges and still be within the scope of this disclosure. It is also contemplated that the thickness of a slit 103 in a body facing liner 28 material in the absorbent article 10 in a stretched, laid flat configuration can vary as compared to the thickness of a slit 103 in a body facing liner 28 during or prior to the manufacturing of the absorbent article 10, due to considerations including, but not limited to, stretch in the body facing liner 28. However, as previously noted, the measurements of the characteristics of the intersecting slit formations 102 described herein are measured when the absorbent article 10 is in a stretched, laid flat configuration, such as that shown in FIG. 2.

As mentioned above, an intersecting slit formation 102 can include an aperture 104. The aperture 104 of the intersecting slit formation 102 can be circular in shape, however, similar to the shape of the intersecting slits 103 discussed above, the aperture 104 can be a different shape, including, but not limited to, elliptical, polygonal (triangular, rectangular, etc. . . . ), or irregularly shaped. The aperture 104 can be of various dimensions. For example, a circular shaped aperture 104 as depicted in FIG. 8 can have a diameter selected from the range of 0.5-10.0 mm, more preferably from the range of 0.8-7.0 mm, and even more preferably from the range of 0.9-2.5 mm. Of course, an aperture 104 can be sized such that it is outside this exemplary range.

FIG. 8 also illustrates the potential open area 109 for an intersecting slit formation 102. The potential open area 109 is illustrated with a dash-dot-dash broken line in FIG. 8 that is configured by constructing a perimeter around the intersecting slit formation 102 by connecting the distal end 103b of each successive slit 103 with a linear segment. The potential open area 109 of an intersecting slit formation 102 can approximate the potential open area in the horizontal plane of the body facing liner 28 for a particular intersecting slit formation 102 that can allow fluid and/or particulate exudates to pass through the body facing liner 28 without having to physically pass through the body facing liner 28 material itself. A sum of the total potential open areas 109 of each intersecting slit formation 102 of a pattern 106 can define a total potential open area of the body facing liner 28. In some embodiments, the total potential open area can be between about 1% to about 70% of the total area of the body facing liner 28, more preferably can be between about 3% to about 50% of the total area of the body facing liner 28, and even more preferably can be between about 10% to about 40% of the total area of the body facing liner 28.

Referring back to FIG. 7, the body facing liner 28 can include a plurality of intersecting slit formations 102. The plurality of intersecting slit formations 102 can be designed to form a pattern 106 on the body facing liner 28. The pattern 106 can be rectangular in shape, hourglass in shape, circular, elliptical, polygonal, or any other desired shape. The pattern 106 of intersecting slit formations 102 can extend throughout the body facing liner 28, from longitudinal side edge 18 to longitudinal side edge 20 and from front waist edge 22 to rear waist edge 24. Alternatively, the pattern 106 of intersecting slit formations 102 can be concentrated such that the pattern 106 does not extend to one or more longitudinal side edge 18, 20 and one or more waist edge 22, 24, as shown in FIG. 7. Alternatively, the plurality of intersecting slit formations 102 can form no repeated pattern at all, and be located randomly on the body facing liner 28.

In a preferred embodiment, intersecting slit formations 102 in the body facing liner 28 can be provided to substantially align and correspond with the apertures 76 in the acquisition layer 70. Furthermore, the pattern 106 of the intersecting slit formations 102 can be provided to substantially align and correspond with the pattern 78 of the apertures 76 in the acquisition layer 70. FIG. 7 presents one example of how a pattern 106 of the intersecting slit formations 102 and can be provided to substantially align and correspond with the pattern 78 of the apertures 76 in the acquisition layer 70. FIG. 7 illustrates a body facing liner 28 overlaying an acquisition layer 70. The body facing liner 28 includes a pattern 106 of intersecting slit formations 102 that includes seven rows 107 of intersecting slit formations 102 and three columns 108 of intersecting slit formations 102. The acquisition layer 70, which is the acquisition layer 70 depicted in FIG. 3, includes seven rows 80 of apertures 76 and three columns 82 of apertures 76 (labeled in FIG. 3). Accordingly, there is the same amount of intersecting slit formations 102 in the body facing liner 28 as there are apertures 76 in the acquisition layer 70 in this exemplary embodiment.

The potential open area 109 for a majority of the intersecting slit formations 102 in the body facing liner 28 can at least partially overlap a portion of the open area 88 of a corresponding aperture 76 in the acquisition layer 70. As used in this context herein, “overlaps” refers to the comparative position of the potential open area 109 and the open area 88 in the longitudinal and lateral directions 30, 32, respectively, when viewed from the vertical direction 34 perpendicular to both the longitudinal and lateral directions 30, 32, (i.e., perpendicular to the plane of the body facing liner 28 when the body facing liner 28 is laid flat). In some embodiments, such as that depicted in FIGS. 7 and 8, the potential open area 109 for a majority of the intersecting slit formations 102 in the body facing liner 28 can be completely within the open area 88 of a corresponding aperture 76 in the acquisition layer 70. In some embodiments, the intersection point 105 of a majority of the intersecting slit formations 102 is within the open area 88 of the corresponding aperture 76, the term “within” used in this context referring to the comparative position of the intersection point 105 and the open area 88 when viewed from the vertical direction 34 perpendicular to both the longitudinal and lateral directions 30, 32, (i.e., perpendicular to the plane of the body facing liner 28 when the body facing liner 28 is laid flat). In some embodiments, such as the embodiment shown in FIGS. 7 and 8, the intersection point 105 of a majority of intersecting slit formations 102 can substantially align with the center point 89 of each corresponding aperture 76. As used in the context herein, “substantially aligns” refers to the comparative position of the intersection point 105 and the center point 89 when viewed from the direction perpendicular 34 to both the longitudinal and lateral directions 30, 32, (i.e., perpendicular to the plane of the body facing liner 28 when the body facing liner 28 is laid flat).

Configuring the body facing liner 28 and the acquisition layer 70 such that intersecting slit formation(s) 102 in the body facing liner 28 align or correspond to aperture(s) 76 in the acquisition layer 70 can provide benefits for the absorbent article 10. For example, fluid and/or particulate matter that passes through the potential open area 109 of an intersecting slit formation 102 in the body facing liner 28 can flow more quickly to the absorbent body 35 by passing through the open area 88 of the aperture 76 in the acquisition layer 70. Such an alignment can help reduce the area of spread of an insult on the body facing liner 28 and reduce the residual fecal matter on the body facing liner 28 after an insult of exudates. As a result, skin irritation of the wearer of the absorbent article 10 can also be reduced by this alignment. Additionally, such enhanced properties individually, as well as collectively, can reduce the likelihood of the fluid and/or particulate matter exudates from compromising the gasketing system of the absorbent article 10, such as the containment flaps 44, 46.

While some of the advantages of exudate distribution and control can be further enhanced when a fluid acquisition layer 70 having channels 90 and apertures 76 is used in combination with a body facing liner 28 including a plurality of intersecting slit formations 102 as described above, it is contemplated that the fluid acquisition layers 70, 170, 270, 370, 470, 570, 670 can be used in conjunction with various other body facing liners 28, including body facing liners 28 with no intersecting slit formations 102. Other body facing liners 28 suitable for use in the absorbent article 10 can include body facing liners 28 known by those of skill in the art and can include body facing liners 28 with or without apertures.

The body facing liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the body facing liner 28. The body facing liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The body facing liner 28 need not be a unitary layer structure, and thus, can include more than one layer of fabrics, films, and/or webs, as well as combinations thereof. For example, the body facing liner 28 can include a support layer and a projection layer that can be hydroentagled.

For example, the body facing liner 28 can be composed of a meltblown or spunbond web of polyolefin fibers. Alternatively, the body facing liner 28 can be a bonded-carded web composed of natural and/or synthetic fibers. The body facing liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire body facing liner 28 or it can be selectively applied to particular sections of the body facing liner 28.

In an embodiment, a body facing liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a body facing liner 28 can be a spunbond substrate with a basis weight from about 10 or 12 to about 15 or 20 gsm. In an embodiment, a body facing liner 28 can be a 12 gsm spunbond-meltblown-spunbond substrate having 10% meltblown content applied between the two spunbond layers.

Although the backsheet 26 and body facing liner 28 can include elastomeric materials, it is contemplated that the backsheet 26 and the body facing liner 28 can be composed of materials which are generally non-elastomeric. In an embodiment, the body facing liner 28 can be stretchable, and more suitably elastic. In an embodiment, the body facing liner 28 can be suitably stretchable and more suitably elastic in at least the lateral or circumferential direction of the absorbent article 10. In other aspects, the body facing liner 28 can be stretchable, and more suitably elastic, in both the lateral and the longitudinal directions 32, 30, respectively.

Containment Flaps:

In an embodiment, containment flaps, 44, 46, can be secured to the body facing liner 28 of the absorbent article 10 in a generally parallel, spaced relation with each other laterally inward of the leg openings to provide a barrier against the flow of body exudates. In an embodiment, the containment flaps, 44, 46, can extend longitudinally from the front waist region 12 of the absorbent article 10, through the crotch region 16 to the back waist region 14 of the absorbent article 10. The containment flaps 44, 46 can be bonded to the body facing liner 28 with adhesive or other means as are known in the art. Alternatively, each containment flap 44, 46 can be bonded to other components of the absorbent article 10 other than the body facing liner 28, including, but not limited to, the backsheet 26.

The containment flaps, 44 and 46, can be constructed of a fibrous material which can be similar to the material forming the body facing liner 28. Other conventional materials, such as polymer films, can also be employed. Each containment flap, 44 and 46, can include flap elastics, such as flap elastics 48 and 50, respectively. Suitable elastic materials for the flap elastic, 48 and 50, can include sheets, strands or ribbons of natural rubber, synthetic rubber, or thermoplastic elastomeric materials. The flap elastics, 48 and 50, as illustrated, can have two strands of elastomeric material extending longitudinally along the containment flaps, 44 and 46, in generally parallel, spaced relation with each other. The elastic strands can be within the containment flaps, 44 and 46, while in an elastically contractible condition such that contraction of the strands gathers and shortens the containment flaps, 44 and 46. As a result, the elastic strands can bias the containment flaps, 44 and 46, toward a position spaced from a position of where the containment flaps, 44 and 46, are bonded to absorbent article 10 such that a portion of the containment flaps, 44 and 46, can extend away from the body facing liner 28 in a generally upright orientation of the containment flaps, 44 and 46, especially in the crotch region 16 of the absorbent article 10, when the absorbent article 10 is fitted on the wearer. The containment flaps, 44 and 46, can be connected to the flap elastics, 48 and 50, by partially doubling a portion of the containment flap, 44 and 46, material back upon itself by an amount which can be sufficient to enclose the flap elastics, 48 and 50. It is to be understood, however, that the containment flaps, 44 and 46, can have any number of strands of elastomeric material and may also be omitted from the absorbent article 10 without departing from the scope of this disclosure.

Leg Elastics:

Leg elastic members 56, 58 can be secured to the backsheet 26, such as by being bonded thereto by laminate adhesive, generally laterally inward of the longitudinal side edges, 18 and 20, of the absorbent article 10. The leg elastic members 56, 58 can form elasticized leg cuffs 57, 59, respectively, that further help to contain body exudates. In an embodiment, the leg elastic members 56, 58 may be disposed between the inner layer 62 and outer layer 60 of the backsheet 26 or between other layers of the absorbent article 10. The leg elastic members 56, 58 can be a single elastic member as illustrated in the figures herein, or each leg elastic member 56, 58 can include more than one elastic member. A wide variety of elastic materials may be used for the leg elastic members 56, 58. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

Fastening System:

In an embodiment, the absorbent article 10 can include a fastener system. The fastener system can include one or more back fasteners 130 and one or more front fasteners 132. Portions of the fastener system may be included in the front waist region 12, back waist region 14, or both. The fastener system can be configured to secure the absorbent article 10 about the waist of the wearer and maintain the absorbent article 10 in place during use. In an embodiment, the back fasteners 130 can include one or more materials bonded together to form a composite ear as is known in the art. For example, the composite fastener may be composed of a stretch component 134, a nonwoven carrier or hook base 136, and a fastening component 138.

Waist Elastic Members:

In an embodiment, the absorbent article 10 can have waist elastic members, 52 and 54, which can be formed of any suitable elastic material. The waist elastic member 52 can be in a rear waist region 12 of the absorbent article 10 and the waist elastic member 54 can be in a front waist region 14 of the absorbent article 10. Suitable elastic materials for the waist elastic members 52, 54 can include, but are not limited to, sheets, strands or ribbons of natural rubber, synthetic rubber, or thermoplastic elastomeric polymers.

The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist elastic members, 52 and 54, may be omitted from the absorbent article 10 without departing from the scope of this disclosure.

Feminine Hygiene Product:

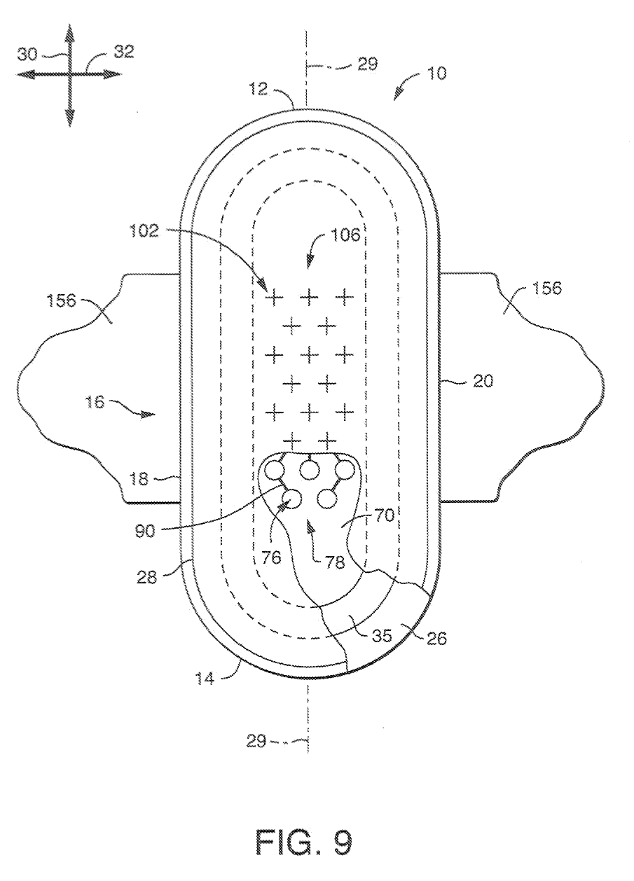

FIG. 9 provides a non-limiting illustration of an absorbent article 10 in the form of a feminine hygiene product such as a menstrual pad or feminine adult incontinence product. Additionally, the absorbent article 10 can include first and second longitudinally opposed front and rear end regions, 12 and 14 (which can be referred to as front waist regions and rear waist regions, respectively), and an intermediate region (or crotch region) 16, located between the end regions, 12 and 14. The absorbent article 10 can have first and second longitudinal side edges, 18 and 20. The longitudinal side edges, 18 and 20, can be contoured to match the shape of the absorbent article 10. The absorbent article 10 can have any desired shape such as, for example, a dog bone shape, a race track shape, an hourglass shape, or the like. Additionally, the absorbent article 10 can be substantially longitudinally symmetric, or may be longitudinally asymmetric, as desired.

As representatively shown, the dimension of the absorbent article 10 in the longitudinal direction 30 can be relatively larger than the transverse dimension of the absorbent article 10 in the lateral direction 32. Configurations of the absorbent article 10 can include a body facing liner 28 and a backsheet 26, such as described herein. An absorbent body 35, such as described herein, can be positioned between the body facing liner 28 and the backsheet 26. As representatively shown, for example, the peripheries of the body facing liner 28 and the backsheet 26 can be substantially entirely coterminous or the peripheries of the body facing material 28 and the backsheet 26 can be partially or entirely non-coterminous. In an embodiment, the absorbent article 10 can include an acquisition layer 70 such as described herein.

The body facing liner 28 can include a pattern 106 of intersecting slit formations 102, such as described herein. The acquisition layer 70 can include a pattern 78 of apertures 76. The acquisition layer 70 can include one or more channels 90, as described herein, to provide advantageous distribution and retention characteristics in the absorbent article 10. The intersecting slit formations 102 in the body facing liner 28 can correspond to and be aligned with the apertures 76 in the acquisition layer 70, as previously described, to provide further advantages for the absorbent article 10 as noted above.

In an embodiment in which the absorbent article 10 can be a feminine hygiene product, the absorbent article 10 can include laterally extending wing portions 156 that can be integrally connected to the side edges, 18 and 20, of the absorbent article 10 in the intermediate region 16 of the absorbent article 10. For example, the wing portions 156 may be separately provided members that are subsequently attached or otherwise operatively joined to the intermediate region 16 of the absorbent article 10. In other configurations, the wing portions 156 may be unitarily formed with one or more components of the absorbent article 10. As an example, a wing portion 156 may be formed from a corresponding, operative extension of the body facing liner 28, the backsheet 26, and combinations thereof. The wing portions 156 can have an appointed storage position (not shown) in which the wing portions 156 are directed generally inwardly toward the longitudinal axis 29. The wing portions 156 can have any operative construction and can include a layer of any operative material. Additionally, each wing portion 156 can comprise a composite material. For example, the wing portions 156 can include a spunbond fabric material, a bicomponent spunbond material, a necked spunbond material, a neck-stretched-bonded laminate (NBL) material, a meltblown fabric material, a bonded carded web, a thermal bonded carded web, a through-air bonded carded web, or the like, as well as combinations thereof.

Each wing portion 156 can include a panel-fastener component (not shown) which can be operatively joined to an appointed engagement surface of its associated wing portion 156. The panel-fastener component can include a system of interengaging mechanical fasteners, a system of adhesive fasteners, or the like, as well as combinations thereof. In an embodiment, either or both wing portions 156 can include a panel-fastener system which incorporates an operative adhesive. The adhesive may be a solvent based adhesive, a hot melt adhesive, a pressure-sensitive adhesive, or the like, as well as combinations thereof.

In an embodiment, a garment attachment mechanism (not shown), such as a garment attachment adhesive, can be distributed onto the garment side of the absorbent article 10. In an embodiment, the garment adhesive can be distributed over the garment side of the absorbent article 10 of the backsheet 26, and one or more layers or sheets of release material can be removably placed over the garment adhesive for storage prior to use. In an embodiment, the garment attachment mechanism can include an operative component of a mechanical fastening system. In such an embodiment, the garment attachment mechanism can include an operative component of a hook-and-loop type of fastening system.

In an embodiment, a chemical treatment may be employed to alter the color of bodily exudates captured by the absorbent article 10. In an embodiment, for example, the treatment may be a decolorizing composition that agglutinates (agglomerates) red blood cells in blood and menses and limits the extent that the red color of menses is visible. One such composition includes a surfactant, such as described in U.S. Pat. No. 6,350,711 to Potts, et al., which is incorporated herein in its entirety by reference thereto. Besides agglutinating agents, the decolorizing composition may alter the chemical structure of hemoglobin to change its color. Examples of such compositions are described in U.S. Patent Application Publication No. 2009/0062764 to MacDonald, et al., which is incorporated herein in its entirety by reference thereto. In an embodiment, the composition can include an oxidizing agent that can be generally capable of oxidizing hemoglobin or other substances responsible for unwanted color of the bodily exudates. The decolorizing composition may be applied to any liquid permeable layer of the absorbent article 10 where it can contact aqueous fluids exuded by the body, such as, for example, menses, such as the body facing liner 28, acquisition layer 70, fluid transfer layer 66, absorbent body 35, backsheet 26, and combinations thereof.

All documents cited in the Detailed Description of the Disclosure are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article including a longitudinal axis and a lateral axis being defined in a horizontal plane parallel to the absorbent article in a laid flat configuration, the absorbent article also including a vertical axis perpendicular to the horizontal plane, the absorbent article comprising:
a body facing liner;
a backsheet coupled to the body facing liner;
an absorbent body positioned between the body facing liner and the backsheet; and
a fluid acquisition layer comprising a body facing surface and a garment facing surface, the fluid acquisition layer being positioned between the body facing liner and the backsheet, the fluid acquisition layer further comprising a plurality of apertures and at least one channel including a length and a depth, the length being defined in the horizontal plane by a distance between a proximal end and a distal end of the channel and the depth being defined in a vertical direction parallel to the vertical axis, the proximal end being connected to a first aperture of the plurality of apertures, the at least one channel configured such that the depth extends from the body facing surface to the garment facing surface, and wherein the depth of the at least one channel is greater than half of a thickness of the fluid acquisition layer along at least a portion of the at least one channel, the thickness of the fluid acquisition layer being defined in the vertical direction.

2. An absorbent article including a longitudinal axis and a lateral axis being defined in a horizontal plane parallel to the absorbent article in a laid flat configuration, the absorbent article also including a vertical axis perpendicular to the horizontal plane, the absorbent article comprising:
a body facing liner;
a backsheet coupled to the body facing liner;
an absorbent body positioned between the body facing liner and the backsheet; and
a fluid acquisition layer positioned between the body facing liner and the absorbent body, the fluid acquisition layer comprising a first layer and a second layer, the first layer including a top surface providing a body facing surface of the fluid acquisition layer and an opposing bottom surface, the second layer including a bottom surface providing a garment facing surface of the fluid acquisition layer and an opposing top surface, the fluid acquisition layer further comprising a plurality of apertures and at least one channel including a length and a depth, the length being defined in the horizontal plane by a distance between a proximal end and a distal end of the channel and the depth being defined in a vertical direction parallel to the vertical axis, the proximal end being connected to a first aperture of the plurality of apertures, the at least one channel configured such that the depth extends through at least one of the first layer and the second layer, and wherein the depth of the at least one channel is greater than half of a thickness of the fluid acquisition layer along at least a portion of the at least one channel, the thickness of the fluid acquisition layer being defined in the vertical direction.

3. The absorbent article of claim 1, wherein the distal end of the at least one channel is connected to a second aperture of the plurality of apertures.

4. The absorbent article of claim 1, wherein the fluid acquisition layer further comprises a plurality of channels.

5. The absorbent article of claim 4, wherein at least one aperture is connected to at least two channels of the plurality of channels.

6. The absorbent article of claim 4, wherein a majority of the plurality of channels include a proximal end that is connected to at least one aperture of the plurality of apertures and a distal end that is connected to a different aperture of the plurality of apertures.

7. The absorbent article of claim 2, wherein the fluid acquisition layer further comprises a plurality of channels, at least one of the plurality of channels being in the first layer and at least one of the plurality of channels being in the second layer.

8. The absorbent article of claim 1, wherein the depth of the at least one channel is the same along the length of the at least one channel.

9. The absorbent article of claim 1, wherein the depth of the at least one channel varies along the length of the at least one channel.

10. The absorbent article of claim 1, wherein the body facing liner includes a plurality of intersecting slit formations.

11. The absorbent article of claim 10, wherein a majority of the plurality of intersecting slit formations of the body facing liner include a potential open area that at least partially overlap with corresponding apertures of the plurality of apertures of the fluid acquisition layer.

12. The absorbent article of claim 10, wherein each of the plurality of intersecting slit formations of the body facing liner include an intersection point and each of the apertures defines an open area in the fluid acquisition layer, and wherein the intersection point for a majority of the plurality of intersecting slit formations is within corresponding open areas of the plurality of apertures of the fluid acquisition layer.

13. The absorbent article of claim 2, wherein the at least one channel extends all the way through the first layer from the body facing surface of the fluid acquisition layer to the bottom surface of the first layer.

14. The absorbent article of claim 2, wherein the at least one channel extends all the way through the second layer from the top surface of the second layer to the garment facing surface of the fluid acquisition layer.

15. The absorbent article of claim 2, wherein the at least one channel extends all the way through the first layer and the second layer from the body facing surface of the fluid acquisition layer to the garment facing surface of the fluid acquisition layer.

16. The absorbent article of claim 2, wherein the bottom surface of the first layer is bonded directly to the top surface of the second layer.

17. The absorbent article of claim 1, wherein the plurality of apertures extend through the fluid acquisition layer from the body facing surface of the fluid acquisition layer to the garment facing surface of the fluid acquisition layer.

18. The absorbent article of claim 2, wherein the plurality of apertures extend completely through the one of the first layer and the second layer having the at least one channel.

19. The absorbent article of claim 1, wherein the fluid acquisition layer includes a channeled region including a plurality of channels and a non-channeled region free from channels.

20. The absorbent article of claim 1, further comprising:
a front waist region, a rear waist region, a crotch region, the crotch region being disposed between the front waist region and the rear waist region; and
a front waist edge in the front waist region, a rear waist edge in the rear waist region, a first longitudinal side edge and a second longitudinal side edge, the first longitudinal side edge and the second longitudinal side edge each extending from the front waist edge to the rear waist edge.
